(19) United States
(12) Patent Application Publication
(10) Pub. No.: US 2024/0269434 A1
(43) Pub. Date: Aug. 15, 2024

(54) ADJUSTABLE SELF-ANCHORING CATHETERS AND METHODS OF USE

(71) Applicant: PAVmed, Inc., New York, NY (US)

(72) Inventors: Lishan Aklog, New York, NY (US); Brian J. deGuzman, New York, NY (US); Richard Yazbeck, New York, NY (US); Michael Boutillette, New York, NY (US)

(21) Appl. No.: 18/561,451

(22) PCT Filed: May 31, 2022
(86) PCT No.: PCT/US22/31567

§ 371 Date: Nov. 16, 2023

Related U.S. Application Data
(60) Provisional application No. 63/196,354, filed on Jun. 3, 2021.

(51) Int. Cl. A61M 25/02

(52) U.S. Cl. CPC A61M 25/02; A61M 2025/0286; A61M 2025/0293

(57) ABSTRACT

A self-anchoring catheter is provided that includes a body, an anchor portion, and an actuator. The body having a proximal end and a distal end, and defining a pathway therebetween, the body being axially rigid. The anchor portion can be imparted on the body to secure the body to surrounding tissue. The actuator can be disposed adjacent the proximal end of the body to positionally control movement of the anchor portion and its position relative to the actuator.

### CROSS-REFERENCE TO RELATED APPLICATION

This application claims priority to, and the benefit of, U.S. Provisional Application No. 63/196,354, filed Jun. 3, 2021, for all subject matter common to both applications. The disclosure of said provisional application is hereby incorporated by reference in its entirety.

### TECHNICAL FIELD

The present disclosure generally relates to catheters such as percutaneous catheters. The present disclosure relates, in particular, to the use of an adjustable anchoring section for self-anchoring catheters within tissue.

### BACKGROUND

A wide variety of catheters can be inserted into patients for short-term and long-term use. These catheters can be inserted into different types of anatomic structures including vascular structures (e.g., veins, arteries, cardiac chambers), body cavities and spaces (e.g., thoracic, pericardial, peritoneal, epidural, thecal) and visceral organs (e.g., stomach, intestines, bladder). They are used for various purposes including infusion of substances (e.g., fluids, medications, blood products, nutritional), withdrawal of blood or other bodily fluids for diagnostic or therapeutic purposes (e.g., drainage, decompression), monitoring of physiologic parameters (e.g., pressure, temperature) and as a conduit through which therapeutic or diagnostic instruments are passed.

Catheters commonly used for percutaneous applications include Percutaneous Venous Catheters (PVCs), Central Venous Catheters (CVCs) and Peripherally Inserted Central Catheters (PICCs). PVCs are inserted through the skin into a peripheral vein, usually in the arm, and are the most common means of delivering fluids or medications into patients. CVCs are inserted through the skin into a central vein and usually remain in place for a long period of time, especially when the reason for their use is longstanding. PICCs are traditionally placed adjacent the antecubital fossa and advanced to the superior vena cava, subclavian vein, or other suitable location to deliver IV medications. PICCs can stay in the body for weeks or months, alleviating the need to subject your veins to the numerous needle sticks. PVCs, CVCs and PICCs are secured into positions utilizing various means. For example, CVCs are sometimes inserted in more critical locations, and the catheters are sutured to the skin and frequently have eyelets, suture guides or other features to facilitate suturing. Other catheters are secured using simple or elaborate taping schemes. There are a wide variety of proprietary catheter anchoring devices being marked which uses a variety of adhesives, straps, and other mechanisms.

Catheter dislodgment is an issue for a variety of reasons. Inadvertent dislodgement of certain catheters such as CVCs, chest tubes, large arterial sheaths and others can lead to serious complications including air embolism, pneumothorax, hemorrhage or even death. Furthermore, replacing dislodged catheters can expose patients to additional risks of discomfort and infection, interfere with the therapeutic regimen or other care and lead to complications from the reinsertion procedure. The economic burden resulting from dislodged catheters or the various efforts and protocols necessary to prevent dislodgement can be significant.

Accordingly, there is a need for catheters that can be anchored to the skin without a need for suturing, elaborate taping and/or additional anchoring devices. Additionally, there is a need to provide catheters that include adjustable anchoring sections to accommodate different patients, uses and techniques.

### SUMMARY

In some embodiments, a catheter extending between a proximal end and a distal end, the catheter includes a hub disposed adjacent the proximal end, a body extending from the hub to the distal end, the body and the hub defining a pathway extending from the proximal end to the distal end, and an anchoring member moveable relative to at least one of the hub and the body.

In accordance with exemplary embodiments, a self-anchoring catheter is provided for herein. The self-anchoring catheter includes a body having a proximal end and a distal end, and defining a pathway therebetween, the body being axially rigid; an anchor portion imparted on the body to secure the body to surrounding tissue; and an actuator disposed adjacent the proximal end of the body to positionally control movement of the anchor portion and its position relative to the actuator.

In some embodiments, the body can be axially rigid. The anchor portion can be a jacket configured to receive at least a portion of the body. The jacket can include a helical portion that forms an anchoring section with at least one turn. The jacket can further include a linear section, in addition to the at least one turn. The jacket can be configured to reshape a section of the body that is disposed therein.

In some embodiments, the anchor portion can be a stiffened member configured to be disposed within the body. The stiffened member can include a helical portion that forms an anchoring section with at least one turn. The stiffened member can further include a linear section. The stiffened member can be configured to reshape a section of the body that is disposed therein.

In some embodiments, the actuator can include a control interface configured to actuate movement of the anchor portion. The control interface can include a wheel disposed on an outer surface of the actuator. In some embodiments, the body includes a plurality of lumens. The anchor portion can include a plurality of turns at a predefined pitch such that the anchor portion creates a traction force within surrounding tissue. The plurality of turns can include helical turns configured to receive the surrounding tissue. The actuator, in some embodiments, can include an enclosure which at least partially receives the anchor portion in at least one configuration.

In accordance with exemplary embodiments a self-anchoring catheter is provided. The self-anchoring catheter includes a body having an anchor portion thereon imparted with shape memory capability; the body being axially rigid and having a pathway extending from its proximal end through the anchor portion to its proximal end; a transitioning member movable along the body to flatten the anchor portion to provide a substantially straight body and straight pathway; and an actuator disposed adjacent the proximal end of the body to control movement of the transitioning member along the body.

In some embodiments, the transitioning member can include a sheath configured to telescopically receive at least a portion of the body and transition the body to a linear condition. The transitioning member can be a sheath telescopically received over the body, configured to expose portions of the body upon movement of the transitioning member relative to the body, wherein an exposed portion of the body can be configured to return to an anchoring condition. The body, in an anchoring condition, can include a helical shape having at least one turn. The transitioning member can be a stiffened member received within the body, and translatable distally, to prevent the body from forming an anchoring condition.

In accordance with exemplary embodiments, a method for operating a self-anchoring catheter is provided. The method includes inserting a catheter into a tissue, the catheter including a body having a proximal end and a distal end, and defining a pathway therebetween, the body being axially rigid; an anchor portion imparted on the body to secure the body to surrounding tissue; and an actuator disposed adjacent the proximal end of the body to positionally control movement of the anchor portion and its position relative to the actuator; actuating the actuator to translate the anchor portion relative to the actuator; and anchoring the catheter to the tissue using the anchor portion.

In some embodiments, the anchor portion can be a jacket, having a helical portion, disposed over a least a portion of the body, and wherein actuating the actuator can include reshaping a portion of the body to have a helical shape. In some embodiments, the anchor portion can be a stiffened member, having a helical portion, disposed within a least a portion of the body, and actuating the actuator can include reshaping a portion of the body to have a helical shape. The body can be formed from a shape memory material capable of transitioning between a linear condition and an anchoring condition, and actuating the actuator can include moving a transitioning member to allow the body to return to the anchoring condition.

### BRIEF DESCRIPTION OF THE DRAWINGS

Illustrative, non-limiting example embodiments will be more clearly understood from the following detailed description taken in conjunction with the accompanying drawings.

FIGS. 1A-C illustrate embodiments of a catheter having an adjustable anchoring section in the form of a jacket;

FIGS. 2A-C illustrate embodiments of a catheter having an adjustable anchoring section in the form of a stiffened inner member;

FIGS. 2D-J illustrate various example of a multi-lumenal catheter body;

FIGS. 3A-B illustrate embodiments of a catheter having a shape-memory body;

FIGS. 4A-B illustrate embodiments of a catheter having a moveable transitioning section;

FIGS. 5A, 5B, and 5C illustrate certain variations of the helical section;

FIG. 6 illustrates a floating teeth configuration.

### DETAILED DESCRIPTION OF THE EMBODIMENTS

Various exemplary embodiments will be described more fully hereinafter with reference to the accompanying drawings, in which some example embodiments are shown. The present inventive concept may, however, be embodied in many different forms and should not be construed as limited to the example embodiments set forth herein. Rather, these example embodiments are provided so that this disclosure will be thorough and complete, and will fully convey the scope of the present inventive concept to those skilled in the art. In the drawings, the sizes and relative sizes of layers and regions may be exaggerated for clarity. Like numerals refer to like elements throughout.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Embodiments of the present disclosure generally provide self-anchoring catheters and catheter systems for percutaneous applications. The various embodiments of the present disclosure can be used to infuse or withdraw fluids from bodily tissues, and to provide short- or long-term venous accesses.

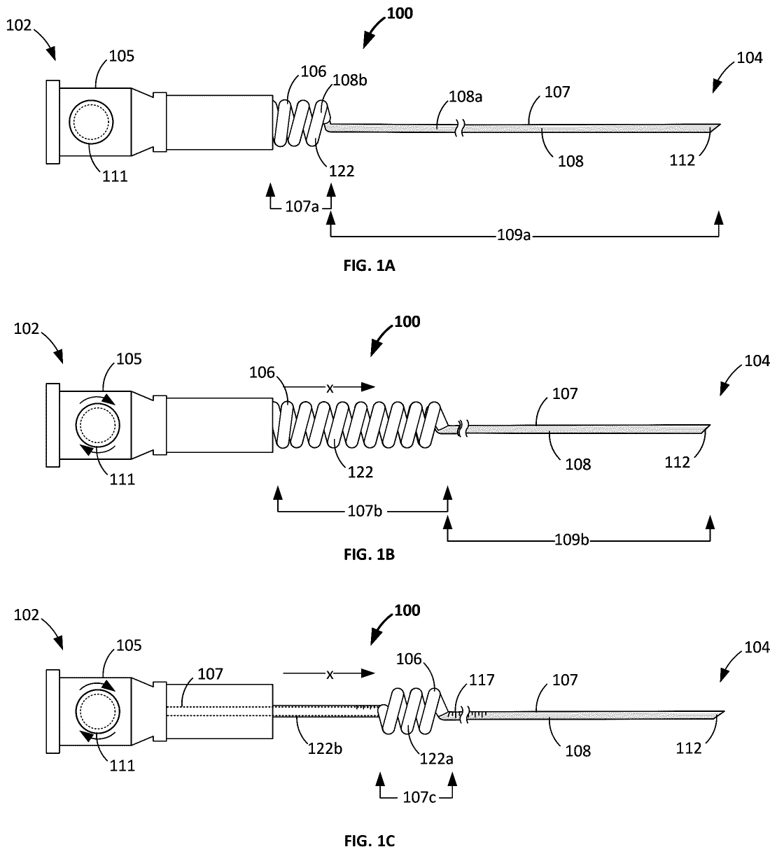

FIGS. 1A and 1B illustrate a catheter 100 in accordance with various embodiments of the present disclosure. Referring to FIGS. 1A-B, the catheter 100 can generally extend between a proximal end 102 and a distal end 104. The catheter can include a hub 105 at the proximal end, an adjustable anchoring section 106 (anchor portion), and a body 107. The body can include one, or more, lumens which can define one or more pathways 108 extending through the length of the catheter 100, e.g., a single lumen as illustrated in FIGS. 1A-B.

In some embodiments, the body 107 can be configured for communicating with anatomic structures. In some examples, the body 107 can be connected to the hub 105 at a fixed distance. The overall length of the body 107 can vary to better accommodate the insertion of the catheter 100 into different types of anatomic structures. In some embodiments, the body 107 may further include a distal tip 112, and the body 107 and/or the distal tip 112 can be placed at desired locations for transporting (i.e., delivering or withdrawing) fluids. In some embodiments, the distal tip 112 can be sufficiently rigid to pierce through tissue to access a desired anatomic structure. In some embodiments, the body 107 may be substantially straight in nature for passing through multiple layers of tissues of an anatomic structure. The body 107 and, specifically, distal tip 112 may be placed at the desired locations, where fluids can be delivered or withdrawn at the distal tip 112 and then through the pathway 108. To better assist the insertion and anchoring of the catheter at the various types of anatomic structures, the body 107 may be constructed to be rigid, semi-rigid or flexible and may possess one or more lumens designed for different types of venous applications. In an embodiment, the body 107 can be axially rigid to allow for insertion into tissue and, at the same time, radially flexible, or malleable. In general, the catheter 100 and its various components may be made from any material that is biocompatible, including, but not limited to, plastic, metal or ceramic.

In some embodiments, as shown in FIGS. 1A-B, for anchoring the catheter 100 at a surgical site, the catheter 100 can include an anchoring section 106 designed to secure the catheter 100 onto a tissue without using, for example, sutures, tapes, or additional anchoring apparatuses. The anchoring section 106 may be designed to be directly connected to, or unitarily formed with, the hub 105. In some examples, anchoring section 106 may be disposed over a portion of body 107 and movable relative to hub 105. Fluids can be transported through the entire length of the catheter 100, from the hub 105 to the distal tip 112, via the pathway 108 which extends through the entire anchoring section 106. Alternatively, fluid may be drained from the distal tip 112 to the hub 105. For the purpose of better assisting the initial insertion into a tissue, the catheter 100 can be equipped with a distal tip 112 that may be sharp and pointed and designed to penetrate tissues. Or, in some embodiments, an integrated needle or an insertion kit can be used to firstly penetrate the tissue and then guide the catheter 100 to the desired anatomic location. The insertion needle can include a proximal needle hub that is connectable to the hub 105 of the catheter 100.

Anchoring section 106 can be constructed in several ways. As shown in FIGS. 1A-B, anchoring section 106 may include a telescoping, substantially rigid, anchoring jacket 122. Anchoring jacket 122 may be configured to translate in and/or out of hub 105, or otherwise move relative thereto, and configured and arranged to accept, cover, or slide around, a section of the body 107 of the catheter 100. To anchor the catheter 100 at a tissue site, the anchoring section 106, and, specifically, anchoring jacket 122, can be curved to assume a corkscrew-like or helical configuration, designed to anchor into tissue structures. This curved or helical structure can include a plurality of turns spaced apart at certain pitch designed to create a traction force with surrounding tissue. Each turn of the helical structure can, in general, have a width that is slightly larger than the diameter of the body 107 so as to receive the body section therein. Dimensions and pitch distances of the anchoring jacket 122 can be configured to optimize the traction between the helical turns and the tissue body. For example, the diameter of the helical portion of anchoring jacket 122 can be substantially larger (e.g., 1.25×, 1.5×, 1.75×, 2×, 2.5×, 3×, 4× or more) than the opening created by the section of the body 107 when the catheter is initially inserted into the tissue, thereby ensuring that the turns of the helical anchoring jacket 122 can be used to secure the surrounding tissues. In an embodiment, the jacket 122 can be fixed relative to the body 107, and the jacket can be movably received within the hub 105. Upon actuation of the catheter 100, the fixed assembly of the jacket 122 and body 107 can translate the anchoring section 106 from within, and thus relative to, the hub 105. In such an embodiment, the distance between distal tip 112 and anchoring section 106 is fixed, and may not change.

It should be appreciated that the length provided to the anchoring jacket 122, in some embodiments, should be sufficient to optimize traction, and that although a helical design is provided, other geometric designs can be implemented, so long as such a design permits that anchoring section to be advanced to secure the device in place. In some embodiments, for a 1 mm diameter catheter, the anchoring jacket 122 can have about one to six full turns, the helix diameter can be about two to six times the catheter diameter (e.g., about 1 to 6 mm), and the pitch between the turns can be about 1 to 4 mm.

The hub 105, as illustrated, may be positioned at a proximal end of the catheter 100 and can be designed to be connected to a wide variety of instruments, such as but not limited to, an infusion source, a withdrawal mechanism, a monitoring device or serve as a portal of entry for diagnostic or therapeutic instruments. To that end, the hub 105 may be of any shape or dimension so long as it can be attached to the desired instrument. The hub 105 can define a housing, or at least a partial opening, which can receive at least a portion of the anchoring jacket 122 and/or the anchoring section 106.

The hub 105 can include an actuating mechanism, or control interface, such as a wheel 111, disposed on hub 105, which may be rotated (e.g., clockwise or counter-clockwise). In other examples, the actuating mechanism can be an electromechanical switch, a pneumatic valve, a slide switch, or other electromechanical actuating mechanisms. In the case of a wheel 111, rotation of the wheel 111 can cause the anchoring portion 106 to rotate and/or telescope with respect to, or extend from, hub 105 in the direction of arrow “x.” Alternatively, a toggle, knob, switch, or other actuating member may be used to adjust the position of the anchoring section, or portions of the catheter 100, or hub 105 or anchoring section 106 may be directly handled and actuated (e.g., rotated) with the operator's hand(s). As anchoring section 106 extends from the hub, it arrives substantially underneath or adjacent at least one layer of tissue (i.e., a layer of skin or soft tissue), such that the plurality of helical turns can generate a traction force sufficient with the tissue. In at least some examples, the wheel 111 may be used to extend the anchoring section 106 into the appropriate location within tissue. Alternatively, the wheel 111 may be used to extend the anchoring section 106 adjacent the tissue, and the final anchoring may be performed by turning the catheter 100, or the anchoring jacket 122, in its entirety, to manually embed the anchoring section within the tissue.

In some examples, leaving at least one helical turn proximal to the anchoring tissue allows the catheter 100 to not only resist dislodgement from a traction force but also prevents the catheter 100 from advancing further into the patient from a pushing force. It should be appreciated that the anchoring section 106 can be of any shape or dimension so long as it can create sufficient traction forces with the surrounding tissues to resist against dislodgement. In some embodiments, once the catheter 100 is secured in place, fluids can be transported through the pathway 108, where a second section 108b of the pathway 108 may be designed to follow the curvatures of the helical portion, such that fluids flows through each turn within the helical portion. The second section 108b of the pathway 108 can be entirely housed within the turns of the helical portion and in direct communication with the first section 108a of the pathway 108. The anchoring section 106 as shown in FIGS. 1A-B effectively allows tissues to be lodged between each turns of the helical portion, thereby optimizing the traction force between the catheter 100 with the surrounding tissues. Furthermore, the length and diameter of the helical anchoring portion 106, as well as the number of turns and the pitch distance between turns, can be optimized to better anchor the catheter in different anatomic structures. It should be appreciated that although only one anchoring section 106 is provided, to the extent that certain applications are contemplated, the device can be provided with multiple anchoring sections 106 spaced apart by one or more linear section(s). The availability of multiple anchoring sections can assist in securing the catheter 100 across an area with different tissues. For example, a catheter 100 may include two or more anchoring sections for anchoring the catheter in two different anatomic layers (e.g., skin and fascia). The helical corkscrew-like configuration of the anchoring section 106 can be formed in a variety of ways. In order to serve its anchoring role, the anchoring section 106 may be configured to resist straightening during application of a traction force. As such, the anchoring jacket 122 forming anchoring section 106 may be designed to possess some rigidity. In some embodiments where the distal straight portion of the body 107 is substantially flexible, the anchoring section and, specifically, the anchoring jacket 122 may be constructed from a single piece by treating the helical portion in such a way to render it more rigid than the body or by altering the material as it is being created (e.g., during an extrusion).

As shown in FIGS. 1A and 1B, anchoring section 106 initially only covers a first portion of body 107 so that the helical anchoring configuration is only formed in helical body section 107a. As wheel 111 is turned (or another actuating member is advanced, translated, toggled, or deployed), anchoring section 106, and, specifically, the anchoring jacket 122 begins to move in the direction of arrow “x” and extend out of hub 105, covering more of body 107 and reshaping a portion of the body so that the helical body section 107b is larger than helical body section 107a. Additionally, the distance between distal tip 112 and anchoring section 106 is reduced from a first length 109a to a second length 109b with the advancement of the helical jacket.

Alternatively, in a variation shown in FIG. 1C, the anchoring jacket 122 can include a helical section 122a and a linear section 122b. In an embodiment, the linear section 122b can be adjacent the hub 105, and turning wheel 111 may drive the jacket out of, or relative to, hub 105, so that the helical section 122a covers a selectable, predetermined portion of catheter body 107c and creates the anchoring section 106 at a certain position while leaving a linear section 122b proximal thereto. This configuration may be useful in situations where the user wishes to choose where the anchoring section 106 will be located relative to body 107, but does not want more than a predetermined number of helical turns or excess turns. For example, using this configuration, two, three, four or more helical turns may be formed in helical section 122a, and those turns may be positioned at the appropriate site of anchoring relative to body 107, the positionability of the helical sections 122a being able to adjust the distance between the distal tip 112 and the helical sections 122a. In this example, a series of markings 117 are also disposed on the catheter to help the operator visualize the depth of the catheter or the position of distal tip 112. It will be understood that markings 117 may include numerals, colors, and/or other indicators and that such markings may be combined with, or disposed on, any of the components described herein on the catheter body, the jacket and/or the anchoring section, etc. While the markings 117 are shown with respect to the embodiment of FIG. 1C, it will be understood that the markings 117 can be employed on any embodiment disclosed herein.

In use, during a catheter anchoring process, the catheter 100 can be firstly inserted through a layer of skin and into an appropriate anatomic structure until the anchoring section 106 (i.e., helical portion) reaches the skin entry point. The anchoring jacket 122 can then be advanced and/or rotated until all or most of the anchoring section 106 became submerged underneath the skin. Subsequently, the catheter 100 can be covered with a simple dressing, where the dressing and additional treatment of the entry point can be performed to prevent inadvertent rotation of the catheter 100. In this manner, for at least the reason that the diameter of the helical anchoring portion 106 is substantially larger than the entry opening in the skin, the anchoring section 106 can resist dislodgement from the tissue in longitudinal direction. In some embodiments, removing the catheter 100 from tissue can include removing the dressing, rotating the wheel 111 (or the catheter or the jacket) in the opposite direction of the insertion rotation until the helical anchoring portion 106 is completely outside the tissue body and then sliding the remaining distal tip 112 of the body 107 out of the patient.

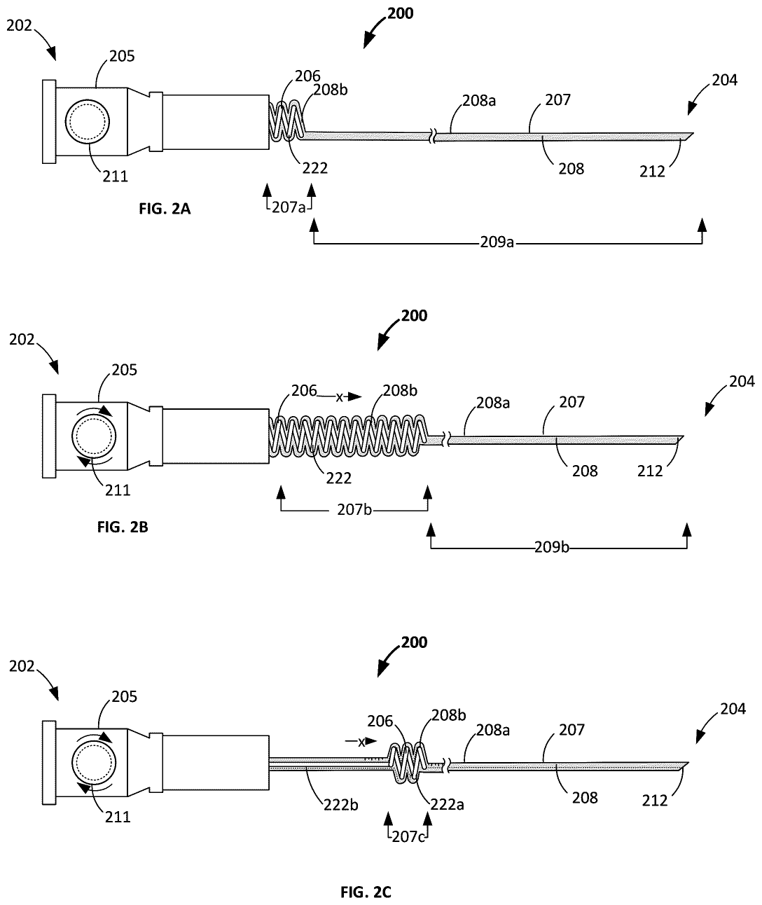

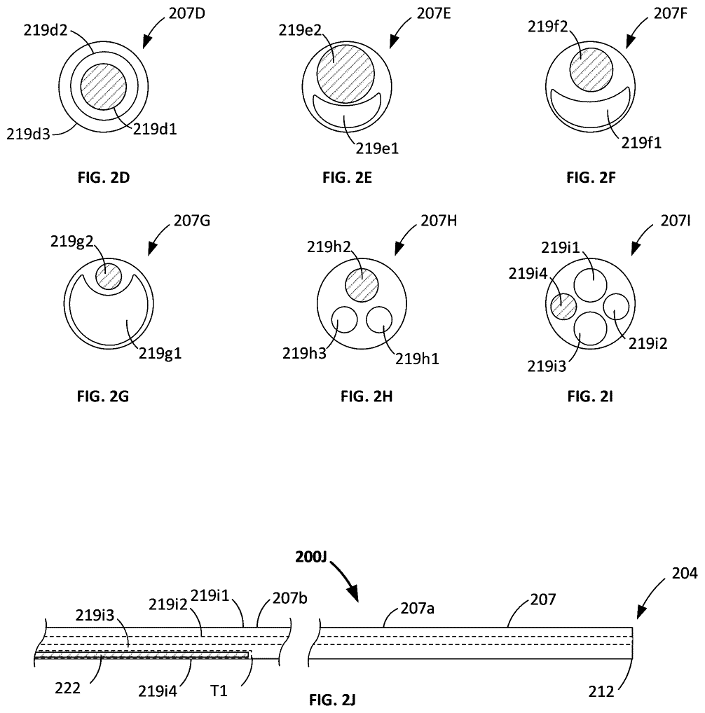

Turning to FIGS. 2A-B, in another embodiment, a catheter 200 may extend between a proximal end 202 and a distal end 204 and include a hub 205 having a wheel 211, a body 207 having a pathway 208 with segments 208a, 208b, and anchoring section 206. The embodiment of FIGS. 2A and 2B can be substantially the same as the embodiments of FIGS. 1A-1C, but for the distinctions discussed herein. For example, instead of an anchoring jacket 122, catheter 200 may include a helical stiffened member 222 that is smaller than pathway 208 and configured and arranged to be disposed therein. In this example, stiffened member 222 may be translatable relative to hub 205 in a manner similar to anchoring jacket 122, but instead of receiving or covering a portion of body 207, it may travel inside pathway 208. Stiffened member 222 may have a helical shape and include a number of turns in the helical shape similar to anchoring jacket 122 including a similar number of turns, measurements, geometries, and/or pitch, etc. As wheel 211 is turned, stiffened member 222 may translate relative to hub 205 (or body 207) in the direction of arrow x and form coils inside body 207 to create the anchoring section 206 at helical body section 207a. As the wheel 211 continues to turn, more of stiffened member 222 can be advanced inside body 207 so that the helical body section 207b is larger or is moved closer to the distal end 204 (FIG. 2B). Additionally, the distance between distal tip 212 and anchoring section 206 is reduced from a first length 209a to second length 209b with the advancement of the stiffened member 222. In some embodiments, the stiffened member 222 can be fixed within the body 207 and the anchoring section 206, defined by the stiffened member 222 within the body 207, can move from within, and relative to, the hub 205.

Similar to FIG. 1C, in one variation, shown in FIG. 2C a stiffened member 222 may include a linear section 222b and a helical section 222a so that a predetermined number of turns in the helical section serve to anchor the catheter body and to effectively choose the position of the anchoring section relative to the body of the catheter to form helical body 207c while avoiding an excess number of turns. For example, using this configuration, two, three, four or more helical turns may be formed in helical section 222a, and those turns may be advanced to the appropriate site of anchoring relative to body 207, the positionability of the helical sections 222a being able to adjust the distance between the distal tip 212 and the helical sections 222a. Markings may also be disposed on the body 207 as previously described.

In some embodiments, the catheter can include a body 207 having a multi-lumenal configuration (e.g., dual lumen or tri-lumen), one for receiving the stiffened member and another or others for delivery and/or withdrawal of fluid, medications, etc. The multi-lumenal configurations may be formed in a concentric arrangement or side-to-side arrangement. FIGS. 2D-I illustrate various bodies 207D-207I of a catheter having one or more lumens (e.g., 219d1, 219d2, 210d3, 219e1, 219e2, 219f1, 219f2, 219g1, 219g2, 219h1, 219h2, 219h3, 219i1, 219i2, 219i3, 219i4). The lumens may be side-by-side (e.g., 219e2, 219e1 of FIG. 2E) or concentric (e.g., 219d1, 219d2, 219d3 of FIG. 2D) as shown, and may take the form of cylindrical tubes having circular cross-sections (e.g., 219d1, 219d2, 219d3) or crescent-shaped tubes of various sizes (e.g., 219e1, 219f1 of FIG. 2F) or combinations thereof. One, two, three, four, five, six, or more lumens may be formed in a body. As shown, certain ones of the lumens are shaded with a diagonal pattern to illustrate that a stiffened member is passing through it to actuate and/or form the anchoring helical section(s). It will be understood that the stiffened member can pass through any of the other lumens, circular or crescent-shaped, and that multiple stiffened members may also be used. The size of a lumen may be chosen to be slightly larger than the stiffened member. Alternatively, the sizes of pathway 208 and stiffened member 222 may be chosen so that a single lumen is required to receive the stiffened member 222 while allowing delivery and/or withdrawal of fluid, medication, blood, saline, etc. All of the lumens may extend through the entire body 207 of the catheter 200. Alternatively, certain one(s) of the lumens may terminate at a position between the proximal and distal ends of the body 207. For example, in FIG. 2J, catheter 200J includes lumen 219i1-219i4, and three of the lumens extend from one end of the body to the other, while lumen 219i4, configured to accept a stiffened member 222, is formed in only one portion of the body 207 near the proximal end, and terminates at a location T1 prior to the distal end, the terminal end of lumen 219i3 being spaced 4, 6, 8, 10, 12, 14 or more centimeters from the distal tip 212. That is, a lumen 219i4 may be chosen specifically for accepting a stiffened member, and the lumen may have a length that prevents the stiffened member from advancing past a certain point to the distal tip 212, the maximum extent being chosen according to the type of catheter and/or the procedure to be performed.

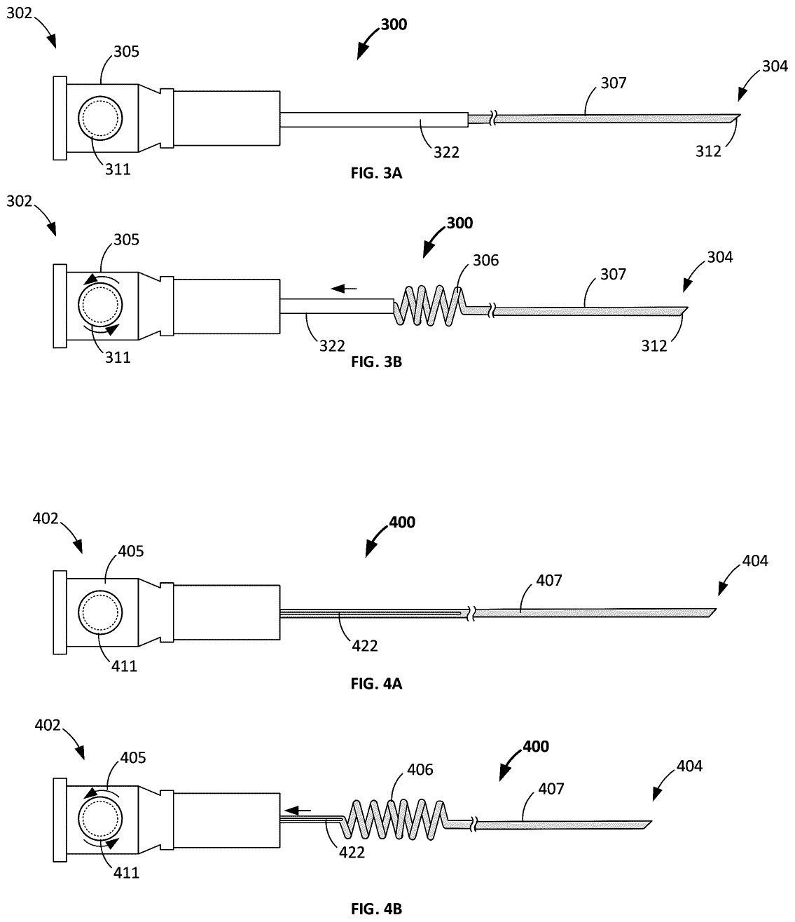

In some embodiments, shown in FIGS. 3A & 3B and 4A & 4B, the body of catheters 300,400 may include one or more helical sections, and additional features may be used to transition the body between linear and anchoring conditions. Like numerals refer to like previously-described elements except that they are preceded by a “3,” or “4,” instead of a “1”. Thus, catheter 300 is similar to catheter 100 and includes a body 307 that is similar to body 107. Specifically, in FIGS. 3A-B, body 307 may be fabricated so that it has a helical anchoring section 306 when no external force is applied thereto. In this example, body 307 may be formed of a shape memory material that is heat-set or otherwise treated to have a helical anchoring section 306. A stiffened sheath 322, having a substantially linear shape, may be applied over this helical section as shown in FIG. 3A so that the catheter 300 is generally linear. To form an anchoring section, the user may turn wheel 311 to retract stiffened sheath 322 (or pull on the sheath directly) into hub 305 to reveal or expose an additional portion of body 307, and to allow a portion of the body to form helical anchoring section 306. Using this technique, any length and/or number of turns may be chosen for the anchoring section by choosing the extent of retraction of stiffened sheath 322 and the part of the body 307 that is exposed. As shown, the distal tip 312 may be proximally drawn upon the formation of the anchoring section. The body 307 can generally extend from a proximal end 302, proximate the hub 305, to a distal end 304.

In a variation, as shown in FIGS. 4A and 4B, catheter 400 includes a hub 405, a catheter body 407 and a stiffened member 422 disposed within a lumen of catheter body 407. The catheter body 407 can generally extend from a proximal end 402, proximate the hub 405, to a distal end 404. Catheter body 407 may be formed to have generally linear section(s) and one or more helical section(s) and may be formed of a shape memory material such that under no external forces, the one or more helical sections are formed. Specifically, catheter body 407 may be capable of transitioning between an anchoring condition having the helical section(s) and a linear condition for insertion and removal. As shown, stiffened member 422 may be initially inserted within a lumen of catheter body 407 and used to prevent a helical section of catheter body 407 from transitioning to its helical rest state, as shown in FIG. 4A. As stiffened member 422 is retracted using wheel 411, or other actuator, the helical anchor section 406 can be re-formed in catheter body 407, due to the shape memory of the material, and the catheter 400 may be anchored within tissue as described in greater detail with some of the other embodiments.

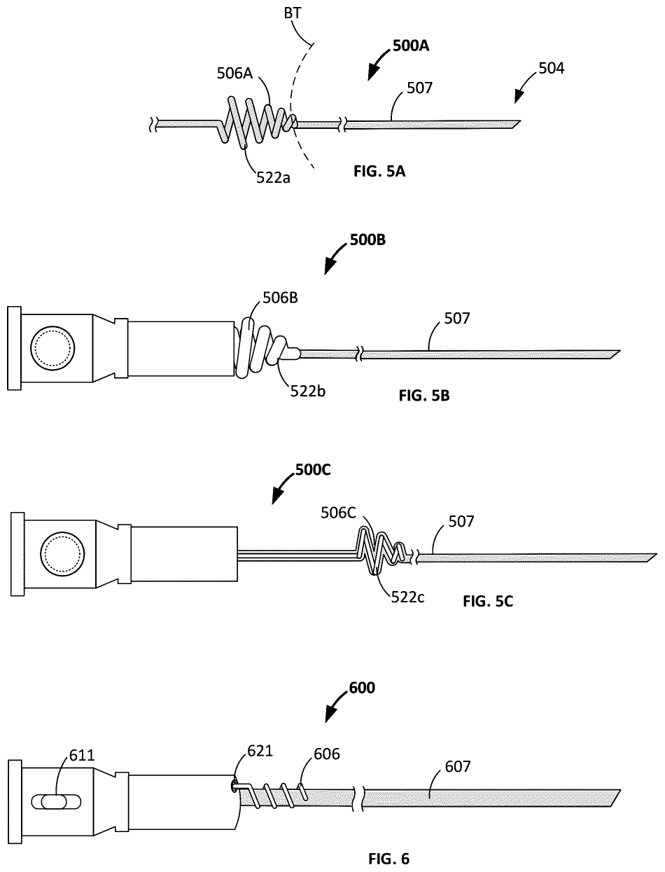

The catheters described so far have included helical anchoring sections of a fixed or constant diameter. In FIG. 5A, one variation is illustrated in which the helical anchoring section 506A of catheter 500A is formed in body 507 of the catheter the helical anchoring section 506A having a tapered series of diameters 522a from a larger, proximal, end to a smaller, distal, end. This may aid in advancing the anchoring portion into body tissue “BT”, beginning from a smaller diameter helical section, nearer to a distal end 504, to progressively larger ones. Instead of forming the tapered helical anchoring section 506A in the catheter body 507, the tapered series of diameters may also be formed in any of the configurations described above, such as in a substantially rigid anchoring jacket 522b to form helical anchoring section 506B of catheter 500B, which can include progressively decreasing diameters from the proximal end to the distal end, as shown in FIG. 5B. The tapered series of diameters may also be formed in a stiffened inner member 522c to form helical anchoring section 506C of catheter 500C, which can include progressively decreasing diameters from the proximal end to the distal end (FIG. 5C).

In another embodiment, as shown in FIG. 6, a catheter 600 can include a body 607, as described above. In this embodiment, the catheter 600 can include a wire 606 which may be advanced out of a window 621 in the hub. The wire 606 can be configured to tightly wrap around the body 607 to form “floating” teeth similar to threads of a screw. The teeth can be formed from the wire 606 and can be wrapped around but removable from the body. Wire 606 may be formed of a shape memory material, such as nitinol, and may be heat set to assume a helical configuration around body 607. In use, the operator may advance the catheter into the body and actuate the wire 606 to pass it through the window and wrap it around the body to form the anchoring section at the appropriate location on body 607. A button 611, or other actuating mechanism, may be pressed to advance more of wire 606 out of window 621, the wire coiling about body 607 as it advance over the body 607 from the proximal end toward the distal end.

In use, during a catheter anchoring process, the catheter 100, 200, 300, 400, 500, 600 can be firstly inserted through a layer of skin and into an appropriate anatomic structure. At such a point, or before insertion into a patient, the actuator (e.g., button 111, 211, 311, 411, or the button 611) can actuate one of the catheter into one of the various anchoring configurations (with the catheter, or the jacket, or the wire). The anchoring section can then be advanced and/or rotated until all or most of the anchoring section becomes submerged underneath the skin. Subsequently, the catheter 100, 200, 300, 400, 500, 600 can be covered with a simple dressing, where the dressing and additional treatment of the entry point can be performed to prevent inadvertent rotation of the catheter. In this manner, for at least the reason that the diameter of the helical anchoring portion 106 is substantially larger than the entry opening in the skin, the anchoring section 106 can resist dislodgement from the tissue in longitudinal direction. In some embodiments, removing the catheter 100 from tissue can include removing the dressing, rotating the wheel, or button, (or the catheter, or the jacket, or the wire) in the opposite direction of the insertion rotation until the helical anchoring portion is completely outside the tissue body and then sliding the remaining distal tip of the body out of the patient. In the case of the embodiment of FIGS. 4A-4B, the helical anchor section 406 of the catheter 400 can be rotated out of the tissue and the stiffened member 422 can be reinserted through the catheter body 407 to straighten the catheter 400. In the case of the embodiment of FIG. 6, the wire 606 can be withdrawn through the window 621 to dislodge the wire 606 from the tissue and allow the catheter 600 to be withdrawn from the tissue.

It should be appreciated that although described as being helical in design or threaded in design, the self-anchoring portion of the catheter can be one of a helical design, a threaded design, any self-anchoring designs, or a combination thereof.

While the present disclosure has been described with reference to certain embodiments thereof, it should be understood by those skilled in the art that various changes may be made and equivalents may be substituted without departing from the true spirit and scope of the disclosure. In addition, many modifications may be made to adapt to a particular situation, indication, material and composition of matter, process step or steps, without departing from the spirit and scope of the present disclosure. All such modifications are intended to be within the scope of the claims appended hereto.

What is claimed is:

1. A self-anchoring catheter comprising:
a body having a proximal end and a distal end, and defining a pathway therebetween, the body being axially rigid;
an anchor portion imparted on the body to secure the body to surrounding tissue; and
an actuator disposed adjacent the proximal end of the body to positionally control movement of the anchor portion and its position relative to the actuator.

2. The self-anchoring catheter of claim 1, wherein the body is axially rigid.

3. The self-anchoring catheter of claim 1, wherein the anchor portion is a jacket configured to receive at least a portion of the body.

4. The self-anchoring catheter of claim 3, wherein the jacket includes a helical portion that forms an anchoring section with at least one turn.

5. (canceled)

6. The self-anchoring catheter of claim 3, wherein the jacket is configured to reshape a section of the body that is disposed therein.

7. The self-anchoring catheter of claim 1, wherein the anchor portion is a stiffened member configured to be disposed within the body.

8. The self-anchoring catheter of claim 7, wherein the stiffened member includes a helical portion that forms an anchoring section with at least one turn.

9. The self-anchoring catheter of claim 8, wherein the stiffened member further includes a linear section.

10. (canceled)

11. The self-anchoring catheter of claim 1, wherein the actuator includes a control interface configured to actuate movement of the anchor portion.

12. (canceled)

13. (canceled)

14. The self-anchoring catheter of claim 1, wherein the anchor portion includes a plurality of turns at a predefined pitch such that the anchor portion creates a traction force within surrounding tissue.

15. The self-anchoring catheter of claim 14, wherein the plurality of turns includes helical turns configured to receive the surrounding tissue.

16. The self-anchoring catheter of claim 1, wherein the actuator includes an enclosure which at least partially receives the anchor portion in at least one configuration.

17. A self-anchoring catheter comprising:
a body having an anchor portion thereon imparted with shape memory capability; the body being axially rigid and having a pathway extending from its proximal end through the anchor portion to its proximal end;
a transitioning member movable along the body to flatten the anchor portion to provide a substantially straight body and straight pathway; and
an actuator disposed adjacent the proximal end of the body to control movement of the transitioning member along the body.

18. The self-anchoring catheter of claim 17, wherein the transitioning member includes a sheath configured to telescopically receive at least a portion of the body and transition the body to a linear condition.

19. The self-anchoring catheter of claim 17, wherein the transitioning member is a sheath telescopically received over the body, configured to expose portions of the body upon movement of the transitioning member relative to the body, wherein an exposed portion of the body is configured to return to an anchoring condition.

20. (canceled)

21. The self-anchoring catheter of claim 17, wherein the transitioning member is a stiffened member received within the body, and translatable distally, to prevent the body from forming an anchoring condition.

22. A method for operating a self-anchoring catheter comprising:
inserting a catheter into a tissue, the catheter including
a body having a proximal end and a distal end, and defining a pathway therebetween, the body being axially rigid;
an anchor portion imparted on the body to secure the body to surrounding tissue; and
an actuator disposed adjacent the proximal end of the body to positionally control movement of the anchor portion and its position relative to the actuator;
actuating the actuator to translate the anchor portion relative to the actuator; and
anchoring the catheter to the tissue using the anchor portion.

23. The method of claim 22,
wherein the anchor portion is a jacket, having a helical portion, disposed over a least a portion of the body, and
wherein actuating the actuator includes reshaping a portion of the body to have a helical shape.

24. The method of claim 22,
wherein the anchor portion is a stiffened member, having a helical portion, disposed within a least a portion of the body, and
wherein actuating the actuator includes reshaping a portion of the body to have a helical shape.

25. The method of claim 22,
wherein the body is formed from a shape memory material capable of transitioning between a linear condition and an anchoring condition, and
wherein actuating the actuator includes moving a transitioning member to allow the body to return to the anchoring condition.
